(19) United States
(12) Patent Application Publication
(10) Pub. No.: US 2021/0393189 A1
(43) Pub. Date: Dec. 23, 2021

(54) A DEVICE AND DIAGNOSTIC METHOD FOR ASSESSING AND MONITORING COGNITIVE DECLINE

(71) Applicant: The Brain Protection Company PTY LTD, Paddington (AU)

(72) Inventors: David Stephen Celermajer, Vaucluse (AU); John Deanfield, Paddington (AU); Anthony Ujhazy, East Lindfield (AU); Zoran Milijasevic, Bayview (AU)

(21) Appl. No.: 17/284,741

(22) PCT Filed: Oct. 11, 2019
(86) PCT No.: PCT/AU2019/051101

§ 371 Date: Apr. 12, 2021

(30) Foreign Application Priority Data
Oct. 12, 2018 (AU) 2018903860

(51) Int. Cl. A61B 5/00; A61B 5/0205; G16H 50/30; G16H 40/67; G16H 50/20; G16H 10/60; G16H 50/70

(52) U.S. Cl. CPC A61B 5/4088; A61B 5/0205; A61B 5/7275; A61B 5/6822; A61B 5/6824; A61B 5/332; G16H 40/67; G16H 50/20; G16H 10/60; G16H 50/70; G16H 50/30

(57) ABSTRACT

A device (10) for assessing a patient's absolute and/or relative risk of cognitive decline and/or dementia, the device (10) comprising: a probe (12) configured to be placed adjacent to a patient's common carotid artery, internal carotid artery or external carotid artery, at least two sensors (101, 102, 104, 106, 108, 110) associated with the probe (12), the sensors being configured to measure one or more of: wave intensity of carotid pulse; wave power of carotid pulse; and pressure waveform of carotid pulse, pulse wave velocity, artery compliance, artery stiffness, artery diameter; micro-emboli count; Heart rate variability; and changes to the eye or retina.

### TECHNICAL FIELD

The present disclosure relates to a device and diagnostic method for assessing and monitoring cognitive decline. However, it will be appreciated by those skilled in the art that the invention may be used in other medical applications.

### BACKGROUND OF THE INVENTION

The heart supplies oxygenated blood to the body through a network of interconnected, branching arteries starting with the largest artery in the body, the aorta. As shown in the schematic view of the heart and selected arteries in FIG. 1, the portion of the aorta closest to the heart is divided into three regions: the ascending aorta (where the aorta initially leaves the heart and extends in a superior direction), the aortic arch, and the descending aorta (where the aorta extends in an inferior direction). Three major arteries branch from the aorta along the aortic arch: the brachiocephalic artery, the left common carotid artery, and the left subclavian artery. The brachiocephalic artery extends away from the aortic arch and subsequently divides into the right common carotid artery, which supplies oxygenated blood to the head and neck, and the right subclavian artery, which predominantly supplies blood to the right arm. The left common carotid artery extends away from the aortic arch and supplies the head and neck. The left subclavian artery extends away from the aortic arch and predominantly supplies blood to the left arm. Each of the right common carotid artery and the left common carotid artery subsequently branches into separate internal and external carotid arteries.

The descending aorta extends downwardly and defines the descending thoracic aorta and subsequently the abdominal aorta before branching into the left and right iliac arteries. Various organs of the body are supplied by arteries which junction with and are supplied by the descending aorta.

During the systole stage of a heartbeat, contraction of the left ventricle forces blood into the ascending aorta that increases the pressure within the arteries (known as systolic blood pressure). The volume of blood ejected from the left ventricle creates a pressure wave, known as a pulse wave, which propagates through the arteries propelling the blood. The pulse wave causes the arteries to dilate. When the left ventricle relaxes (the diastole stage of a heartbeat), the pressure within the arterial system decreases (known as diastolic blood pressure), which allows the arteries to contract.

The difference between the systolic blood pressure and the diastolic blood pressure is the “pulse pressure,” which generally is determined by the magnitude of the contraction force generated by the heart, the heart rate, the peripheral vascular resistance, and diastolic “run-off” (e.g., the blood flowing down the pressure gradient from the arteries to the veins), amongst other factors. High flow organs, such as the brain, are particularly sensitive to excessive pressure and flow pulsatility. Other organs such as the kidneys, liver and spleen may also be damaged over time by excessive pressure and flow pulsatility.

To ensure a relatively consistent flow rate to such sensitive organs, the walls of the arterial vessels expand and contract in response to the pressure wave to absorb some of the pulse wave energy. As the vasculature ages, however, the arterial walls lose elasticity, which causes an increase in pulse wave speed and wave reflection through the arterial vasculature.

Arterial stiffening impairs the ability of the carotid arteries and other large arteries to expand and dampen flow pulsatility, which results in an increase in systolic pressure and pulse pressure. Accordingly, as the arterial walls stiffen over time, the arteries transmit excessive force into the distal branches of the arterial vasculature.

Research suggests that consistently high systolic pressure, pulse pressure, and/or change in pressure over time (dP/dt) increases the risk of dementia, such as vascular dementia (e.g., an impaired supply of blood to the brain or bleeding within the brain). Without being bound by theory, it is believed that high pulse pressure can be the root cause or an exacerbating factor of vascular dementia and age-related dementia (e.g., Alzheimer's disease). As such, the progression of vascular dementia and age-related dementia (e.g., Alzheimer's disease) may also be affected by the loss of elasticity in the arterial walls and the resulting stress on the cerebral vessels. Alzheimer's disease, for example, is generally associated with the presence of neuritic plaques and tangles in the brain. Recent studies suggest that increased pulse pressure, increased systolic pressure, and/or an increase in the rate of change of pressure (dP/dt) may, over time, cause microbleeds within the brain that may contribute to the neuritic plaques and tangles.

Increased pulse pressure is a hallmark of vascular aging, and has recently been identified to be a potential risk factor for cognitive decline and dementia due to its destructive impact on the fragile microvasculature of the brain.

There is research supporting the relationship between high blood pressure in middle age and later cognitive decline or dementia.

Blood pressure is routinely measured and used as an indicator of the presence of various possible underlying conditions. However, blood pressure measurement alone is not a suitable gauge of cognitive decline. This is because a patient's blood pressure may be elevated or varied as a result of various factors which may be unrelated to cognitive decline.

Research also indicates that the presence of glaucoma and/or some observable changes to the eye and retina may be observed in patients with Alzheimer's disease and in people who are in early stage Alzheimer's and also people with higher risk of developing Alzheimer's.

The likely actual cause of brain damage from high pulse pressure is the “intensity” of the carotid wave as it travels forward into the brain. Accordingly, an increase in the amplitude of pulse-generated waves travelling toward the brain could be an important risk factor for later cognitive decline.

Accurately measuring the internal pressure in an artery is currently not possible using non-invasive methods. At present, measurement probes can be placed in or around blood vessels for this purpose, but these procedures are highly invasive.

Wave intensity analysis which requires the measurement of both blood pressure and blood flow changes can be made with large, bulky ultrasound machines intended for hospital use or out-patient use by specialist physicians (eg SSD-5500 Ultrasound system, Aloka, Japan).

The risk of developing dementia or future cognitive decline is currently assessed by a variety of means including algorithms that include a person's age, education level, hypertension status, cholesterol level, body-mass-index and physical activity (eg., CAIDE Risk Score App, Merz Pharmaceuticals GmbH). Kaffashian et al (2013) has compared the CAIDE to the Framingham stroke risk profile (FSRP) and concluded the FSRP is more strongly associated with 10-year cognitive decline.

These current means to assess the risk of cognitive decline are population based and also do not take into account the additional risk factors concerning the state of the particular person's arterial system nor the wave intensity and other characteristics of the blood pressure pulse.

### Object of the Invention

It is an object of the present invention to substantially overcome or at least ameliorate one or more of the above disadvantages, or to provide a useful alternative.

### SUMMARY OF THE INVENTION

In a first aspect, the present invention provides a device for assessing a patient's absolute and/or relative risk of cognitive decline and/or dementia, the device comprising:

a probe configured to be placed adjacent to a patient's common carotid artery, internal carotid artery or external carotid artery, at least two sensors associated with the probe, the sensors being configured to measure one or more of:
wave intensity of carotid pulse;
wave power of carotid pulse; and
pressure waveform of carotid pulse
pulse wave velocity,
artery compliance,
artery stiffness,
artery diameter;
micro-emboli count;
heart rate variability; and
changes to the eye and retina.

The device further preferably comprises a wrist band having one or more sensors communicating with the probe.

The wrist band preferably includes a remote ECG electrode, a blood pressure applanation tonometry sensor and a blood oxygen saturation sensor.

The sensors preferably include one or more Doppler ultrasound sensors and/or Ultrasonic measurement sensors using a wide beam technique, and/or Micro Electro-Mechanical (MEMS) strain gauge and/or acoustic sensors and/or photoacoustic Doppler flowmetry sensors.

The probe is preferably operational in an initial placement mode, where a suitable location is determined relative to the patient's vasculature and an operating mode where the sensors obtain measurements regarding blood flow characteristics from within the vasculature and mechanical properties of the vasculature.

The device further preferably comprises a sensor configured to determine and indicate if the probe is located with excessive pressure against the patient's skin.

The device further preferably comprises a digital display for displaying measurements obtained by the sensors.

The device further preferably comprises an output data cable connectable with a computer.

The device further preferably comprises a wireless data transmitter.

In a second aspect, the present invention provides a method of assessing a patient's absolute and/or relative risk of cognitive decline and/or dementia, the method including the following steps:

locating a probe of a diagnostic device adjacent to the patient's common carotid artery, internal carotid artery or external carotid artery, the probe having at least two sensors;
taking a first measurement with the diagnostic device to obtain primary data relating to one or more of:
   wave intensity of carotid pulse;
   wave power of carotid pulse;
   pressure waveform of carotid pulse;
   pulse wave velocity,
   artery compliance,
   artery stiffness,
   artery diameter;
   heart rate variability;
   micro-emboli count; and
   changes to the eye or retina,
evaluating the measured primary data obtained from the sensors to forecast the patient's absolute and/or relative risk of cognitive decline and/or dementia.

The method further preferably includes the subsequent steps of: taking a second measurement using the diagnostic device at a later point in time and evaluating any differences in the measured parameters between the first and second measurements; and evaluating the measured data obtained from the sensors to forecast the patient's absolute and/or relative risk of cognitive decline and/or dementia.

The step of evaluating the data preferably includes the step of applying a weighting based on patient specific predetermined risk factors.

The risk factors preferably concern medical status and include one or more of: age, sex, obesity, atrial fibrillation status, stroke history, blood pressure, Body Mass Index (BMI), cholesterol level (total and HDL), head injury history, diabetes, Cardiovascular disease (CVD).

The risk factors preferably concern lifestyle and include one or more of: education level, history of smoking, alcohol consumption, exercise frequency and intensity.

The risk factors preferably concern genetics and include one or more of: family history, specific DNA markers.

The risk factors preferably concern patient existing medication including anti-coagulation medication, anti-hypertensives and cholesterol lowering drugs (e.g., statins).

The method further preferably includes the step of comparing the measured data with stored data to compare the patient with a corresponding demographic to evaluate the patient's risk of cognitive decline and/or dementia.

The wrist band (or a finger wrap) preferably includes a remote ECG electrode, a blood pressure applanation tonometry sensor and a blood oxygen saturation sensor.

The probe is preferably operational in an initial placement mode, where a suitable location is determined relative to the patient's vasculature and an operating mode where the sensors obtain measurements regarding flow and/or pressure characteristics from within the vasculature.

The device further preferably comprises a pressure sensor configured to determine and indicate if the probe is located excessively firmly against the patient's skin.

In a third aspect, the present invention provides a device for assessing a patient's absolute and/or relative risk of cognitive decline and/or dementia, the device comprising:
a probe configured to be placed adjacent to a patient's common carotid artery, internal carotid artery or external carotid artery, the probe including the following sensors for obtaining primary data:
   two doppler ultrasound flow sensors for positioning on an artery to provide proximal and distal measurements of blood flow through the artery by bouncing high-frequency sound waves off red blood cells;
   an ECG electrode for calculating heart rate variability; and
   a MEMS strain gauge for assessing tonometry of an arterial pulse of the artery,
the probe being in communication with a processor, a user display and a user input.

The device further preferably comprises an integrated retinal imaging unit having sensors for collecting primary data concerning a patient's eye or retina.

A method of assessing and/or monitoring a patient's risk of cognitive decline preferably includes the steps of:
obtaining primary data using the device described above; inputting secondary data including one or more of age, sex, obesity, atrial fibrillation status, stroke history, blood pressure, Body Mass Index (BMI), cholesterol level (total and HDL), head injury history, diabetes (type 2), Cardiovascular disease (CVD); and
evaluating the patient's risk of cognitive decline by applying a weighting to each input primary data and each input secondary data to generate an overall risk assessment.

### BRIEF DESCRIPTION OF THE DRAWINGS

A preferred embodiment of the invention will now be described by way of specific example with reference to the accompanying drawings, in which:

FIG. 1 is a schematic illustration of a human heart;

FIG. 2a is a front schematic view of a device for testing cognitive decline;

FIG. 2b is a rear schematic view of a device for testing cognitive decline;

FIG. 3 is a 3-dimensional view of a device to contact both left and right sides of the neck;

FIG. 4 is schematic view of a wrist band for use with the device of FIGS. 2 and 3;

FIG. 5 is a side view of the device positioned in contact with the patient's neck; and

FIG. 6 is a schematic view of an alternative embodiment of a device for testing cognitive decline.

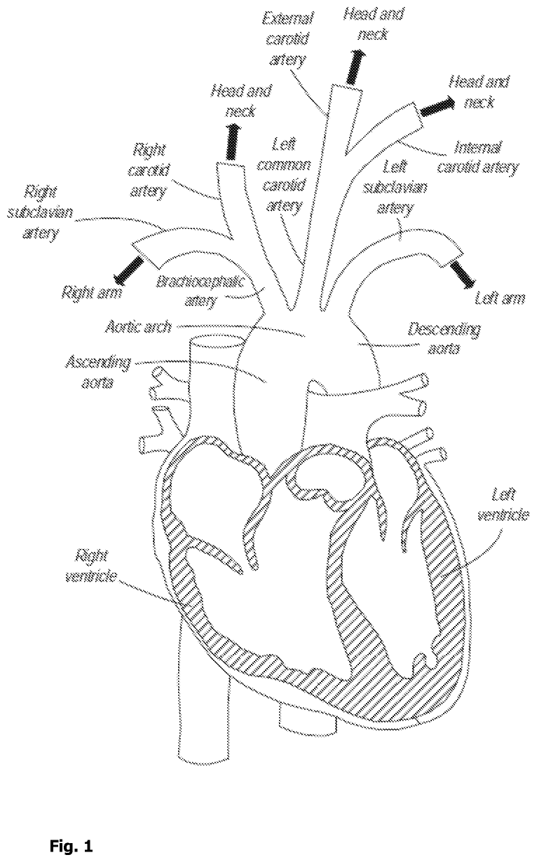

### DETAILED DESCRIPTION OF THE PREFERRED EMBODIMENTS

There is disclosed herein a device 10 and a diagnostic method for testing and monitoring a patient's absolute and/or relative risk of dementia and/or cognitive decline. The device 10 is in particular intended for use in measuring blood flow and/or pressure characteristics within the carotid arteries, including the common carotid artery or the internal carotid artery. It is also intended to measure the biomechanical characteristics of the carotid artery. However, it will be appreciated that the device 10 can also include sensors for taking measurements of other blood vessels, including the vasculature of the retina and eye. Alternatively, data regarding the patient's eyes and/or retinas may be captured separately with a separate imaging apparatus and input into the device 10, or alternatively input into an algorithm based on data obtained by the device 10 and possibly also patient specific risk factors.

The device 10 according to the invention provides an externally applied, non-invasive test which can be used to measure one or more of the following parameters:
dP/dt (change in pressure over time);
pulse pressure;
carotid artery wave intensity;
carotid artery wave power;
pulse wave velocity;
blood flow rate;
blood velocity;
micro-emboli count;
arterial compliance;
arterial stiffness;
arterial diameter and change in diameter with the cardiac cycle;
Heart rate variability;
detectable changes in the eye and retina.

dP/dt provides a good indicator of the rate of upstroke of the pulse and relates in part to left ventricular contractility.

Carotid artery wave intensity relates to the forward moving compression wave.

Pulse wave velocity is the velocity at which the arterial pulse propagates through the circulatory system. Pulse wave velocity provides an indication of arterial stiffness.

Device

The device 10 overcomes the drawbacks associated with current large and bulky ultrasound machines by providing a hand held, compact and operator assistive device to be used in primary care, family care general medical practice facilities. The device 10 may be provided in different configurations such as a probe 12 which is placed against the user's neck, or alternatively as a cuff 14 which is placed around the neck.

In one embodiment, the device 10 includes a probe 12 having an inbuilt microprocessor 15 and software as well as user operated controls and an integrated user display screen 13 or other such digital display which is associated with the device 10. In an alternative embodiment, the device 10 includes a probe 12 which is either connected by a cable or connected wirelessly (eg Bluetooth™) to a separate computational device (eg laptop, PC or mobile phone) running a software application and which also provides both display screen 13 and/or additional user input controls. The remote computational device may also provide power to the probe 12, or the probe may have a self-contained power source (eg rechargeable battery). It will be appreciated by those skilled in the art that the above mentioned embodiments having the microprocessor 15 inbuilt or external are analogous, and the invention may be embodied in either form.

The probe 12 includes several sensors or measurement means, or an array of sensors including one or more of the following:
A Doppler ultrasound sensor 102. A Doppler ultrasound is a non-invasive test that can be used to estimate the blood flow through a blood vessel by bouncing high-frequency sound waves (ultrasound) off red blood cells.
There are preferably two Doppler ultrasound sensors 102 (or groups of sensors), so that the measurement of the arterial flow properties can be made at proximal and distal regions of the carotid arteries. In a preferred embodiment, there are four Doppler ultrasound sensors 102 so that the measurement of the arterial flow properties can be made in the left and right carotid arteries simultaneously.
Ultrasonic measurement of the volumetric flow rate may include the measurement of the blood velocity profile within the vessel or by using a wide beam technique to reduce the need for high spatial resolution.
Phase locked ultrasound tracking may be used to determine the location of the adjacent artery walls (i.e. posterior and superior artery walls) and hence arterial diameter changes within each cardiac cycle. Accuracy may be improved by gating this measurement with cardiac cycle derived from the measurement of the ECG. This data can be used in the calculation of arterial compliance and stiffness that can be used in the algorithm regarding biomechanical properties and relative health of the artery.

In one embodiment, the blood pressure is calculated using the instantaneous arterial cross-sectional area (calculated from the measured diameter) and the elastic properties of the artery wall. The elastic property of the arterial wall can be determined by calculating the Pulse Wave Velocity (PWV). There are a number of ways to determine PWV, eg Pulse Transit Time or, by using the temporal and spatial derivative of the arterial distension waveform or by using the Volumetric Flow rate (Q) and the arterial cross-sectional area (A) which is known as the QA method. The accuracy of this calculation can be enhanced using an ECG electrode 400 to estimate the reflection free period of the cardiac cycle (i.e. early systole). This can also be determined by the pulse pressure waveform.

The probe 12 may measure the change in pressure with time and the pulse pressure using either ultrasonic measurements (eg the Bramwell-Hill equation which relates blood pressure to changes in cross sectional area of the artery via local estimation of PWV) or by applanation tonometry of the carotid pulse. Tonometry of the arterial pulse could be measured by a Micro Electro-Mechanical (MEMS) strain gauge 104.

Blood velocity may be measured using ultrasonic sensors or by a combination of ultrasound and laser (eg photoacoustic Doppler flowmetry).

Wave Intensity analysis can be performed using the measurements of blood pressure (p), velocity (U) and volumetric flow (Q) by the following relationships (where ‘d’=the first derivative and ‘t’=time):

Wave Intensity=dP/dt×dU/dt

And Wave Power=dP/dt×dQ/dt

As described above, the probe 12 or cuff 14 may have one or more sensors (or sets of sensors or sensor arrays) to simultaneously measure the left and right carotid arteries. Measuring left and right arteries may reveal additional information concerning the relative health of the patient in order to further optimise the predictive power of the algorithm.

The probe 12 may include an additional ultrasound sensor 106 to detect micro-emboli.

The probe 12 may have an ECG electrode 400, to be used in conjunction with a second, remote electrode.

The probe 12 may have an acoustic sensor 108 tuned to selectively detect respiration sounds. This could be used to correct calculations (eg wave intensity) for unwanted variations introduced by respiration.

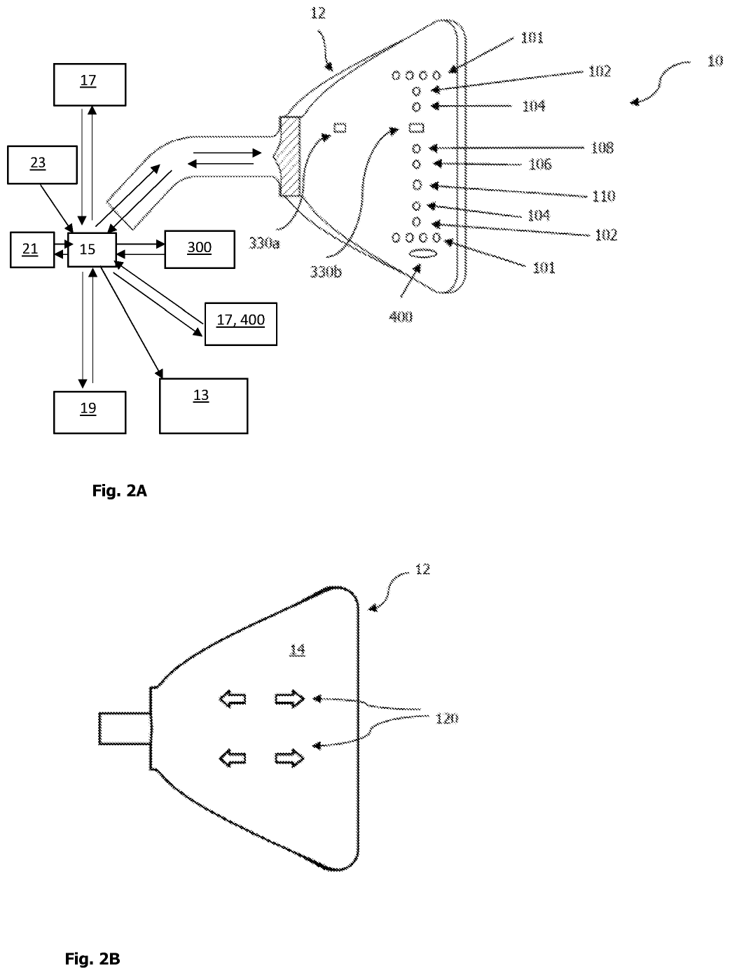

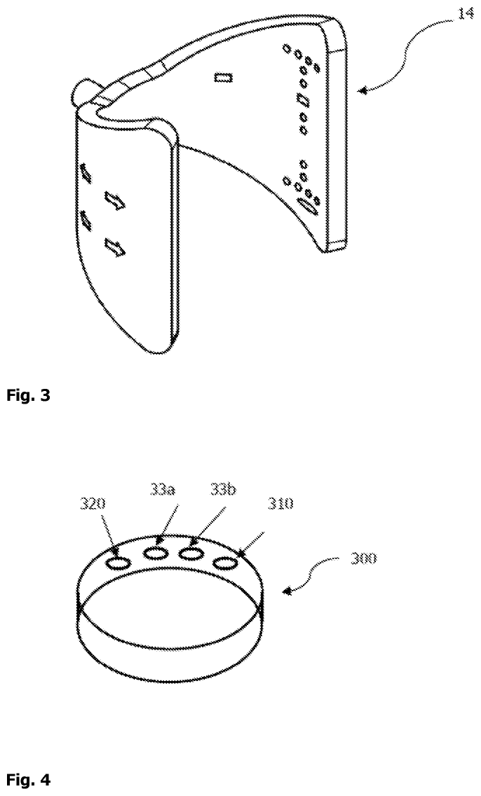

As depicted schematically in FIG. 2A, the probe 12 or cuff 14 may include an internal processor 15 and digital display 13 for displaying measurements obtained by the sensors. The probe also includes buttons, a touch pad or another such user interface 23 for permitting the user to communicate with the processor 15, and control the operation of each sensor included in or associated with the probe 12.

Alternatively, the probe 12 may include an output data cable selectively connectable with a computer having a remote digital display 13. Alternatively, the probe 12 may include a wireless transmitter to remotely transfer the measured data to a remote processor 15, and associated digital display 13 and user interface 23. For example, the probe 12 may have a Bluetooth™ transmitter for communicating with a software application installed in a mobile telephone, tablet, computer or other such processor 15. Alternatively, the probe 12 or cuff 14 may include an integrated micro-processor 15 for storing the data obtained from the probe 12 and using that data with a software-based algorithm to calculate cognitive decline risks based on the parameters measured. The processor 15 includes a memory to store the test results. This may be temporary, or alternatively the test result may be stored for later comparison with subsequently obtained results, for example, conducted 6 or 12 months later.

The device 10 may also have the facility to accept additional inputs from remote sensors, such as second ECG electrode 17 (eg placed on opposite shoulder or side of patient's chest relative to the probe 12 placement); a blood pressure cuff 19 (eg brachial artery) measurement or an applanation tonometry probe placed on the radial artery. In one embodiment depicted in FIG. 4, the device 10 includes remote ECG electrode 310, a blood pressure applanation tonometry sensor 320 and a blood oxygen saturation sensor (light source 33a and detector 33b or alternatively a reflectance pulse oximetry sensor) integrated into a wrist band 300. Accordingly, the device 10 may include a hand-held neck probe, a wrist band and processor 15 in the form of a mobile phone, tablet, PC or integrated processor 15 communicating via Bluetooth, cable connection, infra-red communication, or another data transfer protocol.

The ECG measurement, taken by electrode 400 and/or electrode 17 can also be used to calculate heart rate variability. The frequency spectrum of heart rate includes both a high frequency (HF) and a low frequency (LF) component. Low levels (compared to control populations) of both LF and HF spectral components have been linked to Alzheimer's disease (AD). This measure can be included in the algorithm to model cognitive decline in a specific individual.

Additional remote sensor inputs may include Transcranial Doppler (TCD) and other types of Doppler ultrasonography which may be used to measure the velocity of blood flow through the brain's blood vessels by measuring the echoes of ultrasound waves.

Data from one or more of the above-mentioned remote sensors can be included in an algorithm calculation to improve the sensitivity and or specificity of the cognitive function.

Detectable Changes to the Eye and Retina.

There are potentially four measurements that can be made with respect to the eye and retina, and one additional disease which may be associated with Alzheimer's disease and/or cognitive decline. While the definitive pathology of Alzheimer's disease occurs in the brain, the disease has also been reported to affect the eye, which can be imaged more easily and non-invasively as compared to the brain. A specific type of cataract has been associated with Alzheimer's disease, and a number of retinal changes, including the presence of retinal beta-amyloid plaques, have also been linked to the disease. There is some homology between the retinal and cerebral vasculatures, and the retina also contains nerve cells and fibres that form a sensory extension of the brain. The eye is the only place in the body where vasculature or neural tissue is available for non-invasive optical imaging.

One or more of the following measurements may be made to identify detectable changes in the eye and retina, which may be associated with Alzheimer's disease and/or cognitive decline.
beta-amyloid (hallmark of Alzheimer's disease) accumulation in the retina is obtained by direct retinal imaging. Recent studies have shown that retinal accumulation mirrors that in the brain and importantly possibly earlier than in the brain;
cortical visual impairment has also been linked to Alzheimer's disease;
subretinal Drusen deposits, an accumulation between the Bruch's membrane and the retinal pigment epithelium. Imaging technology can be used to determine Drusen deposits by analysis of the geometry of Fundus reflectance.
Pupil response to light flash: The ocular pupil controls retinal illumination and responds dynamically to a bright flash of light by rapid constriction followed by re-dilation over a longer time period. Pupillometry investigates this response by delivering a flash of light directed into the eye and accurately detecting and measuring pupil size changes over time. Pupillometry has been used to identify a cholinergic deficiency, detected as a change in the constriction phase of the pupil flash response, in Alzheimer's disease.

The device 10 may include an ocular imaging device 21 capable of measuring one or more of the above changes to the patient's retina or eye.

In addition to the four above noted detectable changes to the eye and retina, glaucoma has been associated with Alzheimer's disease and may be included in the medical status portion of the algorithm, discussed below, i.e., inputting whether a patient has a history of glaucoma.

Positioning and Alignment of the Probe Over the Carotid Artery

There are several methods that can be used to aid the clinician in optimising placement of the probe 12 over the artery. The preferred location would be to align the axis of the probe 12 centrally along the long axis of the artery.

The probe 12 may have two operational modes: one being a positioning mode of the probe 12 over the artery and the second mode is data measurement. In the positioning mode, the sensors may automatically determine the quality of the signal(s) and indicate to the user either on the probe 12 (eg, different colour LEDS or arrows, 120) or on the remote device (eg mobile phone screen) which direction to move or twist the probe 12 for acceptable positioning.

For example, the ultrasound sensors 102 on the probe may measure the blood velocity profile (higher at the axial centre of the artery) or measure the diameter of the artery. The MEMS strain gauge 104 may be configured as a near field acoustic sensor and determine acoustic maxima. Once positioning is satisfactory, the probe 12 switches to measurement mode (Alternatively the user may have a control to initiate the measurement mode).

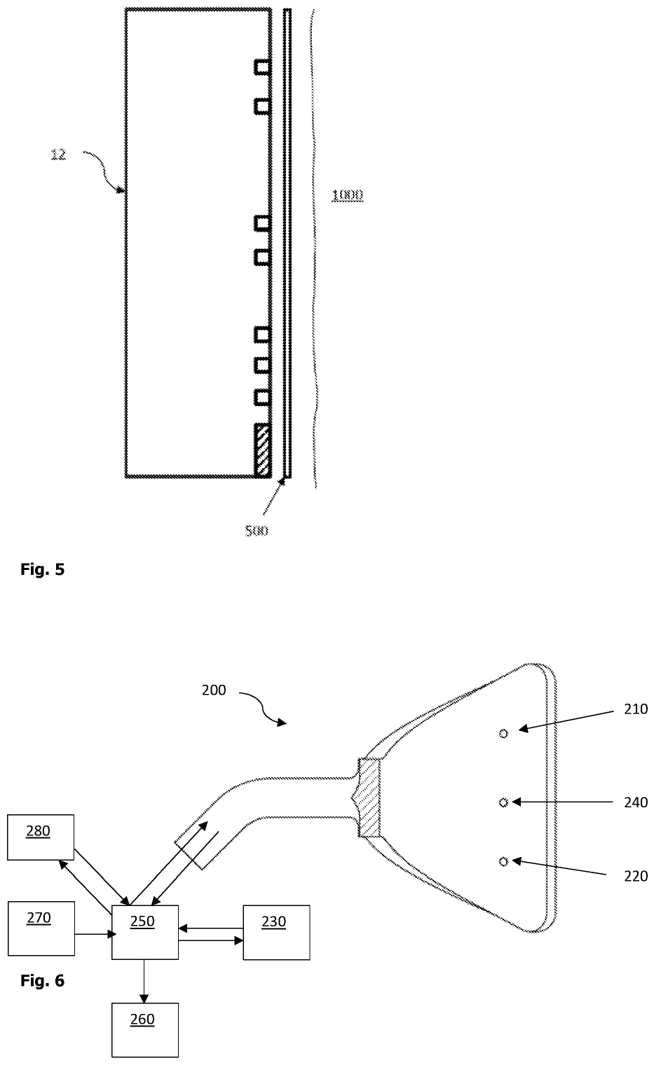

Referring to FIG. 5, the sensors of probe 12 or cuff 14 may be effectively coupled to the patient's skin 1000 (to exclude air) via a single use (per patient, disposable) conforming, gel membrane 500. This may be used instead of semi-liquid gel that is commonly used during ultrasound procedures.

Alternatively, the probe 12 may have an array of sensors 101 as shown in FIG. 2A. The sensor array 101 has multiple sensors of each type and hence has redundant sensors, one or more of which, depending on its position in relation to the artery, would have a higher quality signal than another. The processor 15 and software in the probe 12 (or auxiliary device, eg mobile phone/tablet) calculates which sensor(s) to use for the recording of measurements based on a signal optimisation algorithm derived from the signal strength and quality from each sensor in the array of sensors.

The device 10 may have an additional pressure sensor 110 to detect if the probe 12 is being pushed too hard against the patient's neck such that deformation would likely occur to the carotid artery and potentially cause unwanted changes/errors in measurements of blood flow dynamics. If this is detected, an audible and/or visible alarm may be triggered.

Patient Specific Risk Factors

In order to assess a patient's risk of cognitive decline, there are several types of data which should be considered across a number of areas, primary data being determined by sensor measurements obtained from the device 10, and other secondary data being lifestyle or hereditary in nature. An algorithm can be used to input both the primary and secondary data to assess a patient's personal risk profile, and forecast their individual risk of cognitive decline:
Carotid artery blood flow dynamics: dP/dt, pulse pressure, wave intensity, wave power, PWV (primary data as identified and measured with the device 10);
Carotid artery bio mechanics: carotid artery compliance, carotid artery stiffness (primary data as identified and measured with the device 10);
micro-emboli count (primary data as identified and measured with the device 10);
Medical status: age, sex, obesity, atrial fibrillation status, stroke history, blood pressure, Body Mass Index (BMI), cholesterol level (total and HDL), head injury history, diabetes (type 2), Cardiovascular disease (CVD) presence of glaucoma. (Secondary data obtained from patient's medical record);
Lifestyle: education level, history of smoking, alcohol consumption, exercise frequency and intensity (Secondary data obtained from patient's medical record);
Genetic: family history, specific DNA markers (Secondary data obtained from patient's medical record);
Medications: eg., anti-coagulation medication, anti-hypertensives, cholesterol lowering drugs (e.g., statins) (Secondary data obtained from patient's medical record);
Other tests: brain PET scan, brain MRI (Secondary data obtained from patient's medical record);

An algorithm is used to enter both the primary data and the secondary data, and provide an assessment of risk and/or cognitive decline.

It will be appreciated by those skilled in the art that either the primary data or the secondary data may have a greater weighting in the algorithm, and as such the primary data obtained by the device 10 is not necessarily more important than the secondary data.

Secondary data factors such as medication and exercise level (higher level) are included in the algorithm that may lower the risk of cognitive decline.

Some of the secondary data in the form of medical, lifestyle and genetic factors are included in standardised instruments such as the CAIDE Risk Score (Cardiovascular Risk Factors, Aging, and Incidence of Dementia).

An embodiment of the device 10 is depicted in FIG. 6. In that embodiment, the probe 200 includes some of the aforementioned sensors, as follows:

Two doppler ultrasound flow sensors 210, 220 located for positioning on the same artery to give proximal and distal measurements, preferably along the carotid arteries. The Doppler ultrasound flow sensors 210, 220 provide a non-invasive test that can be used to estimate the blood flow through a blood vessel by bouncing high-frequency sound waves (ultrasound) off red blood cells.

The probe 200 includes an ECG electrode 230 to calculate heart rate variability.

In addition, the probe 200 includes a MEMS strain gauge 240 (for tonometry). Tonometry of the arterial pulse is measured by the Micro Electro-Mechanical (MEMS) strain gauge 240.

The doppler ultrasound flow sensors 210, 220 and ECG electrode 230 provide primary data, which is obtained directly by the device 10.

In accordance with the aforementioned embodiments, the probe 200 is in communication with a processor 250, a user display 260 and a user input 270. The processor 250, user display 260 and user input 270 may be internal or external (wireless or cable connected).

In this embodiment, secondary data in the form of medical status may also be conveyed to the device 10.

The secondary data may include medical status: age, sex, obesity, atrial fibrillation status, stroke history, blood pressure, Body Mass Index (BMI), cholesterol level (total and HDL), head injury history, diabetes (type 2), Cardiovascular disease (CVD).

In addition, retinal imaging results (with respect to changes to the eye or retina) may be obtained directly by the device 10 by way of an integrated retinal imaging unit 280. Alternatively, retinal imaging results may be obtained by a separate test, using existing retinal testing equipment, and the results conveyed to the processor 250 for modelling of risk, in the manner described below, which is relevant to each embodiment.

Modelling Risk of Cognitive Decline

The device 10 is used to measure the aforementioned blood flow and arterial characteristics including dP/dt, artery wave intensity, pulse wave velocity, artery compliance, artery stiffness, and micro-emboli count. The patient may be categorised as high risk or low risk of cognitive decline based on the test results if the primary data in the form of measured parameters obtained by the device 10 are above or below a predetermined level. The primary data may be considered in isolation, or the primary data may be combined with the secondary data to further improve the quality of the modelling and the accuracy of the results and assessment of cognitive decline.

A further test (or plurality of tests) using the device 10 may be subsequently conducted at a later time to assess the changes in the primary data regarding blood flow characteristics (and any of the other characteristics described above). For example, the patient may be tested with the device 10 every 6 months to determine the changes of each of the measured primary data parameters over time. If one or more of the measured parameters reaches and exceeds a predetermined level (or increased above a certain rate over consecutive tests), the patient may be characterised as high risk of cognitive decline. In contrast, if one or more of the measured parameters varies by a predetermined amount or percentage over a period of time, the patient may be characterised as high risk of cognitive decline.

An assessment of the patient's risk of cognitive decline can be made based on the primary data obtained by the device 10. In addition, the patient's personal risk factors may also be factored in to further customise the result. For example, if a patient has a history of smoking, statistically, their risk of cognitive decline will be increased. Accordingly, this customisation can be made in numerous different ways. For example, the measured blood flow characteristics may be altered by multiplying by a variable depending on the presence of certain risk factors. For example, positive risk factors such as exercise could result in a multiplier of less than one, and negative risk factors such as the presence of hereditary cognitive decline could result in a multiplier of more than one.

Alternatively, a score card type assessment may be made where the results tested by the device 10 are entered and allocated a value. In addition, different weightings are applied based on the presence of various positive or negative risk factors, along with patient specific factors such as age, weight, gender etc. This way the results measured by the device 10 can be customised for a specific patient's personal attributes. This weighting of the results enables the data obtained to more accurately predict the risk of cognitive decline for a given patient.

It will be appreciated by those skilled in the art that various algorithms may be employed to assess the patient's absolute and/or relative risk of dementia and/or cognitive decline based on the measurements obtained by the device 10 (primary data) and factoring in the patient specific risk factors (Secondary data).

A software program or application may be used to obtain an indication of the patient's absolute and/or relative risk of cognitive decline and/or dementia based on the readings measured by the device 10 (primary data) and combined with the patient's personal data and risk factors (secondary data).

Furthermore, the patient's measured data (primary data) and risk factors (secondary data) may be compared against a database of stored patient data, or hypothetical patient data to assess the patient's absolute and/or relative risk of cognitive decline and/or dementia.

Results of applicable population-based studies may be incorporated into the algorithm at a later date to improve the sensitivity and/or specificity of the algorithm to a particular person.

### EXAMPLES

1) Hypothetical results for a patient who is characterised as high risk of cognitive decline based on a single test using the device 10.

For example, a person who scores high on the CAIDE risk score and was measured with a high dP/dt (400 mmHg/second or greater) and high carotid wave intensity would be classified as very high risk of cognitive decline.

Conversely a person who had a low risk score on the CAIDE and had a very high dP/dt and high carotid wave intensity would be classified as moderate to high risk of cognitive decline, who should have routine annual re-testing.

2) Hypothetical results for a patient who is characterised as high risk of cognitive decline based on two (or more) tests using the device 10 over a period of time. For example, the measured dP/dt has increased by 30% over 12 months and their arterial stiffness has increased by 20%

3) A 50 year old female with very high pulse pressure, dP/dt and carotid wave intensity and type 2 diabetes (since age 45) may be classified as high risk of cognitive decline.

Treatment

Once a patient has been tested by way of the aforementioned single testing process using the device 10, (or recurring testing over a period of time), if the patient is allocated as falling into a risk category for cognitive decline, a medical intervention may be recommended. This may include prescribing a pharmaceutical preparation. Alternatively, an intra-vascular or extra-vascular device may be operatively placed in or around one or more of the patient's blood vessels to alter the patient's blood flow characteristics. Some examples of such devices are described in the applicant's earlier published international PCT patent application PCT/AU2016/050734.

Although the invention has been described with reference to specific examples, it will be appreciated by those skilled in the art that the invention may be embodied in many other forms.

1. A device for assessing a patient's absolute and/or relative risk of cognitive decline and/or dementia, the device comprising:
a probe configured to be placed adjacent to a patient's common carotid artery, internal carotid artery or external carotid artery,
at least two sensors associated with the probe, the sensors being configured to measure one or more of:
wave intensity of carotid pulse;
wave power of carotid pulse; and
pressure waveform of carotid pulse
pulse wave velocity,
artery compliance,
artery stiffness,
artery diameter;
micro-emboli count;
heart rate variability; and
changes to the eye or retina.

2. The device of claim 1 further comprising a wrist band having one or more sensors communicating with the probe.

3. The device of claim 2, wherein the wrist band includes a remote ECG electrode, a blood pressure applanation tonometry sensor and a blood oxygen saturation sensor.

4. The device of claim 1, wherein the sensors include one or more Doppler ultrasound sensors and/or Ultrasonic measurement sensors using a wide beam technique, and/or Micro Electro-Mechanical (MEMS) strain gauge and/or acoustic sensors and/or photoacoustic Doppler flowmetry sensors.

5. The device of any one of the preceding claims wherein the probe is operational in an initial placement mode, where a suitable location is determined relative to the patient's vasculature and an operating mode where the sensors obtain measurements regarding blood flow characteristics from within the vasculature and mechanical properties of the vasculature.

6. The device of any one of the preceding claims further comprising a sensor configured to determine and indicate if the probe is located with excessive pressure against the patient's skin.

7. The device of any one of the preceding claims further comprising a digital display for displaying measurements obtained by the sensors.

8. The device of any one of the preceding claims further comprising an output data cable connectable with a computer.

9. The device of any one of the preceding claims further comprising a wireless data transmitter.

10. A method of assessing a patient's absolute and/or relative risk of cognitive decline and/or dementia, the method including the following steps:
locating a probe of a diagnostic device adjacent to the patient's common carotid artery, internal carotid artery or external carotid artery, the probe having at least two sensors;
taking a first measurement with the diagnostic device to obtain primary data relating to one or more of:
wave intensity of carotid pulse;
wave power of carotid pulse;
pressure waveform of carotid pulse;
pulse wave velocity,
artery compliance,
artery stiffness,
artery diameter;
micro-emboli count ;
heart rate variability; and
changes to the eye or retina,
evaluating the measured primary data obtained from the sensors to forecast the patient's absolute and/or relative risk of cognitive decline and/or dementia.

11. The method of claim 10, including the subsequent steps of:
taking a second measurement using the diagnostic device at a later point in time and evaluating any differences in the measured primary data between the first and second measurements; and
evaluating the measured data obtained from the sensors to forecast the patient's absolute and/or relative risk of cognitive decline and/or dementia.

12. The method of claim 10 or 11, wherein the step of evaluating the data includes the step of applying a weighting based on secondary data in the form of patient specific predetermined risk factors.

13. The method of claim 12, wherein the secondary data concerns medical status and includes one or more of:
age, sex, obesity, atrial fibrillation status, stroke history, blood pressure, Body Mass Index (BMI), cholesterol level (total and HDL), head injury history, diabetes, Cardiovascular disease (CVD), presence of glaucoma.

14. The method of claim 12, wherein the secondary data concerns lifestyle and includes one or more of: education level, history of smoking, alcohol consumption, exercise frequency and intensity.

15. The method of claim 12, wherein the secondary data concerns genetics and includes one or more of: family history, specific DNA markers.

16. The method of claim 12, wherein the secondary data concerns patient existing medication including anti-coagulation medication, anti-hypertensives and cholesterol lowering drugs (e.g., statins).

17. The method of any one of claim 10 or 16, further including the step of comparing the measured primary data and/or secondary data with stored data to compare the patient with a corresponding demographic to evaluate the patient's risk of cognitive decline and/or dementia.

18. The device of any one of claims 1 to 9, wherein the probe includes:
an internal processor configured to control the sensors and/or record data obtained from the sensors;
a user interface configured to operate the device; and
a user display device configured to display output from the sensors.

19. The device of any one of claims 1 to 9, wherein probe is connectable by a wireless protocol or a cable connection to:
an external processor configured to control the sensors and/or record data obtained from the sensors;
a user interface configured to operate the device; and
a user display device configured to display output from the sensors.

20. A device for assessing a patient's absolute and/or relative risk of cognitive decline and/or dementia, the device comprising:
a probe configured to be placed adjacent to a patient's common carotid artery, internal carotid artery or external carotid artery, the probe including the following sensors for obtaining primary data:
two doppler ultrasound flow sensors for positioning on an artery to provide proximal and distal measurements of blood flow through the artery by bouncing high-frequency sound waves off red blood cells;
an ECG electrode for calculating heart rate variability; and
a MEMS strain gauge for assessing tonometry of an arterial pulse of the artery, the probe being in communication with a processor, a user display and a user input.

21. The device of claim 20 further comprising an integrated retinal imaging unit having sensors for collecting primary data concerning a patient's eye or retina.

22. A method of assessing and/or monitoring a patient's risk of cognitive decline including the steps of:
obtaining primary data using the device of either of claim 20 or 21;
inputting secondary data including one or more of age, sex, obesity, atrial fibrillation status, stroke history, blood pressure, Body Mass Index (BMI), cholesterol level (total and HDL), head injury history, diabetes (type 2), Cardiovascular disease (CVD); and
evaluating the patient's risk of cognitive decline by applying a weighting to each input primary data and each input secondary data to generate an overall risk assessment.
